(19) United States
(12) Patent Application Publication
(10) Pub. No.: US 2023/0240518 A1
(43) Pub. Date: Aug. 3, 2023

(54) SYSTEM AND METHOD FOR SURGICAL INSTRUMENT TRACKING WITH AUTOFOCUS

(71) Applicant: Visionsense Ltd., Petah Tikva (IL)

(72) Inventors: Doron Aloni, Ganey Tikva (IL); Achia Kronman, Pardes Hannah (IL); Rami Cohen, Misgav Regional Council (IL)

(21) Appl. No.: 17/587,206

(22) Filed: Jan. 28, 2022

(51) Int. Cl. A61B 1/00; A61B 1/05; A61B 1/04; A61B 1/045

(52) U.S. Cl. CPC A61B 1/00188; A61B 1/05; A61B 1/043; A61B 1/045; A61B 1/046

(57) ABSTRACT

An endoscope camera assembly includes a focus group having one or more lenses. The focus group is movable along a light path. The camera assembly also includes a sensor configured to receive light and to capture an image. The camera assembly further includes a controller configured to determine a region of interest in the image, compute contrast of the region of interest, and move the focus group to optimize contrast of the region of interest.

### BACKGROUND

Medical endoscopy is increasingly employing specialized optical imaging techniques, such as fluorescence (i.e., autofluorescence and photodynamic) endoscopy, narrow band imaging and other techniques, for improved visualization and for the detection and diagnosis of diseases. Endoscopic imaging systems that provide specialized imaging modes also operate in a conventional color, or white light, endoscopy mode.

In conventional white light endoscopy, light in the visible spectral range is used to illuminate the tissue surface under observation. Light reflected by the tissue passes through a suitable lens system and is incident on an image sensor built into or attached to the endoscope. The electrical signals from the image sensor are processed into a full color video image which can be displayed on a video monitor or stored in a memory.

In fluorescence endoscopy, fluorescence excitation light excites fluorophores in the tissue, which emit fluorescence light at an emission wavelength, which is typically greater than the excitation wavelength. Fluorescence light from the tissue passes through a suitable lens system and is incident on the image sensor. The electrical signals from the image sensor are processed into a fluorescence video image which can be displayed on a video monitor, either separately or combined with the color video image.

The fluorescence excitation and emission wavelengths depend upon the type of fluorophores being excited. In the case of exogenously applied fluorophores, the band of excitation wavelengths may be located anywhere in the range from the ultraviolet (UV) to the near infra-red (NIR) and the emission wavelength band anywhere from the visible to the NIR. For fluorophores endogenous to tissue, the band of excitation and emission wavelengths are more limited (excitation from the UV to the green part of the visible spectrum, emission from the blue/green light to the NIR).

During endoscopic surgical procedures, surgeons use an endoscopic camera along with various surgical instruments. As the surgical instruments are moved through the surgical site, the surgical instruments go out of focus requiring the surgeon to stop operation and to refocus the endoscopic camera. Thus, there is a need to minimize the disruption of refocusing the endoscopic cameras during surgical procedures.

### SUMMARY

According to one embodiment of the present disclosure, an endoscope camera assembly is disclosed. The endoscope camera assembly includes a focus group having one or more lenses. The focus group is movable along a light path. The camera assembly also includes a sensor configured to receive light and to capture an image. The camera assembly further includes a controller configured to determine a region of interest in the image, compute contrast of the region of interest, and move the focus group to optimize contrast of the region of interest.

Implementations of the above embodiment may include one or more of the following features. According to one aspect of the above embodiment, the controller may be further configured to adjust focus of the region of interest. The controller may be also configured to determine the region of interest by identifying at least one of a surgical instrument, an end effector, or a fiducial marker. The endoscope camera assembly may further include an infrared sensor configured to receive near infrared fluorescent light reflected from a fluorescent material of at least one of the surgical instrument, the end effector, or the fiducial marker. The controller is further configured to determine the region of interest based on the near infrared fluorescent light.

The controller may be additionally configured to identify a plurality of surgical instruments. The controller may be further configured to assign a rank to each surgical instrument of the plurality of surgical instruments. The controller may be also configured to determine the region of interest based on the surgical instrument having a highest rank. The endoscope camera assembly may also include a sliding mechanism coupled to the focus group. The sliding mechanism may include at least one crossed-roller bearing. The endoscope camera assembly may also include a linear actuator coupled to the focus group configured to move the focus group along the sliding mechanism. The linear actuator may be a piezoelectric motor. The endoscope camera assembly may also include a magnet coupled to the focus group and a linear magnetic encoder disposed adjacent the focus group and the magnet. The linear magnetic encoder may be configured to measure a position of the focus group. The controller may be coupled to the linear actuator and the linear magnetic encoder. The controller may be further configured to control the linear actuator based on the measured position of the focus group.

According to another embodiment of the present disclosure, a method for controlling an endoscope camera assembly is disclosed. The method includes receiving light through a focus group having one or more lenses. The focus group is movable along a light path. The method also includes capturing an image at a sensor configured to receive light. The method further includes determining a region of interest in the image. The method additionally includes computing contrast of the region of interest and moving the focus group to optimize contrast of the region of interest.

Implementations of the above embodiment may include one or more of the following features. According to one aspect of the above embodiment, the method may also include moving the focus group to adjust focus of the region of interest. Determining the region of interest further may include identifying at least one of a surgical instrument, an end effector, or a fiducial marker. The method may also include identifying a plurality of surgical instruments. The method may further include assigning a rank to each surgical instrument of the plurality of surgical instruments. Determining the region of interest may be based on the surgical instrument having a highest rank.

According to a further embodiment of the present disclosure, an endoscope camera assembly is disclosed. The endoscope camera assembly includes a focus group having one or more lenses. The camera assembly also includes a sliding mechanism coupled to the focus group and configured to move the focus group along a light path. The camera assembly further includes a linear actuator coupled to the focus group and configured to move the focus group along the sliding mechanism. The camera assembly additionally includes a magnet coupled to the focus group and a linear magnetic encoder disposed adjacent the focus group and the magnet. The linear magnetic encoder is configured to measure a position of the focus group. The camera assembly also includes a sensor configured to receive light and to capture an image and a controller configured to: determine a region of interest in the image; compute contrast of the region of interest; and move the focus group to optimize contrast of the region of interest.

Implementations of the above embodiment may include one or more of the following features. According to one aspect of the above embodiment, the controller may be coupled to the linear actuator and the linear magnetic encoder. The controller may be further configured to control the linear actuator based on the measured position of the focus group.

### BRIEF DESCRIPTION OF THE DRAWINGS

The present disclosure may be understood by reference to the accompanying drawings, when considered in conjunction with the subsequent, detailed description, in which:

FIG. 1 is a perspective view of a light endoscopic imaging system according to an embodiment the present disclosure;

FIG. 2 is a perspective view of an endoscope coupled to a camera assembly according to an embodiment the present disclosure;

FIG. 3 is a longitudinal side, cross-sectional view of the camera assembly of FIG. 2, according to an embodiment the present disclosure;

FIG. 4 is a top, perspective of the camera assembly of FIG. 2, according to an embodiment the present disclosure;

FIG. 5 is a side, cross-sectional, perspective view of the camera assembly of FIG. 2, according to an embodiment the present disclosure;

FIG. 6 is a lateral side, cross-sectional view of the camera assembly of FIG. 2, according to an embodiment the present disclosure;

FIG. 7 is a view of a surgical site captured by the imaging system of FIG. 1 according to an embodiment the present disclosure; and

FIG. 8 is a method for controlling the camera assembly of FIG. 2, according to an embodiment the present disclosure.

### DETAILED DESCRIPTION

Embodiments of the presently disclosed system are described in detail with reference to the drawings, in which like reference numerals designate identical or corresponding elements in each of the several views. In the following description, well-known functions or constructions are not described in detail to avoid obscuring the present disclosure in unnecessary detail. Those skilled in the art will understand that the present disclosure may be adapted for use with any imaging system. As used herein the term “distal” refers to that portion of the instrument, or component thereof, farther from the user, while the term “proximal” refers to that portion of the instrument, or component thereof, closer to the user.

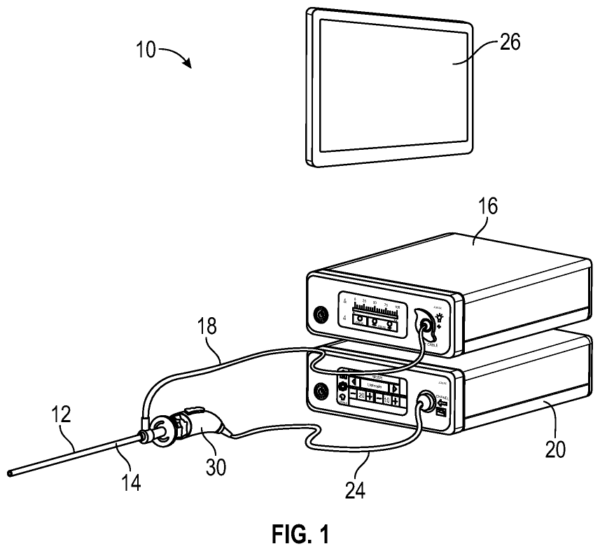

With reference to FIG. 1, an imaging system 10 is configured for combined NIR fluorescence and white light endoscopic imaging. With intraoperative usage of fluorophores from a fluorescent dye, such as indocyanine green (ICG), the imaging system 10 enables real-time visual assessment of blood vessels, lymph nodes, lymphatic flow, biliary ducts, and other tissues during surgical procedures. The imaging system 10 provides an adjunctive method for evaluation of tissue perfusion and related tissue-transfer circulation during surgery. The imaging system 10 may utilize NIR excitation laser light having a wavelength of from about 780 nm to about 805 nm and observation range from about 825 nm to about 850 nm. Fluorescence may be provided by a fluorescent dye having matching excitation and emission ranges. The fluorescence light may be detected by an infrared (IR) channel of a camera assembly to produce an IR image. Other channels of the camera assembly may be used to capture white light images of the same scene. Two images, the white light image and the IR image, may be blended and/or combined to produce a composite image.

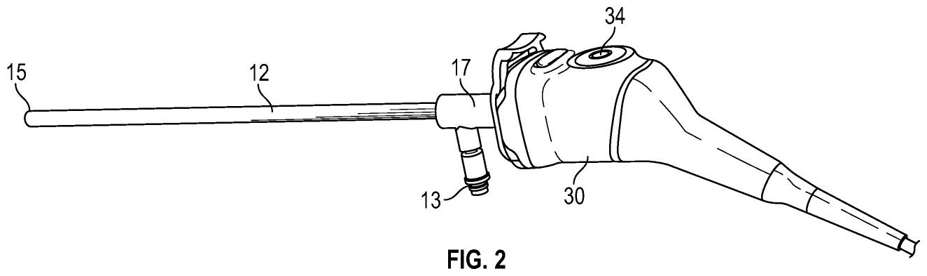

With reference to FIGS. 1 and 2, the imaging system 10 includes an endoscope 12 having a longitudinal shaft 14 with a plurality of optical components (not shown), such as lenses, mirrors, prisms, and the like disposed in the longitudinal shaft 14. The endoscope 12 is coupled to a combined light source 16 via an optical cable 18. The light source 16 may include a white light source (not shown) and an NIR light source (not shown), which may be light emitting diodes or any other suitable light sources. The NIR light source may be a laser or any other suitable light source. The optical cable 18 may include one or more optical fibers for transmitting the white and NIR light, which illuminates the tissue under observation by the endoscope 12. The endoscope 12 collects the reflected white and NIR light and transmits the same to a camera assembly 30, which is coupled to a proximal end portion of the endoscope 12. The endoscope 12 may be any conventional endoscope configured to transmit and collect white and IR light.

The camera assembly 30 is coupled to a camera control unit 20 via a transmission cable 24. The camera control unit 20 is configured to receive the image data signals, process the raw image data from the camera assembly 30, and generate blended white light and NIR images for recording and/or real-time display. The camera control unit 20 also processes the image data signals and outputs the same to a display 26, through any suitable a video output port, such as a DISPLAYPORT™, HDMI®, etc., that can transmit processed images at any desired resolution, display rates, and/or bandwidth.

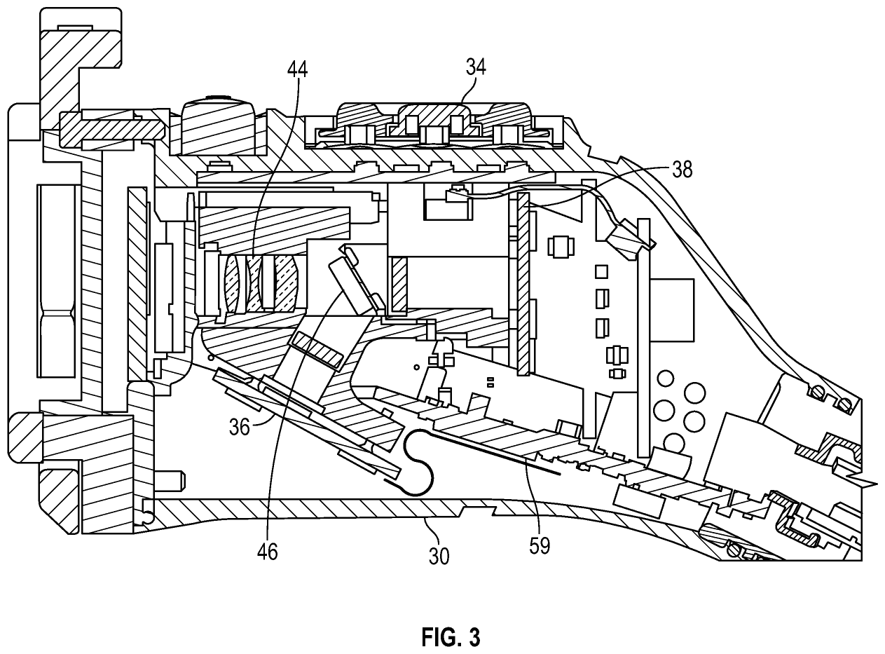

With reference to FIGS. 2 and 3, the endoscope 12 includes a light cable port 13 configured to connect to the optical cable 18. The endoscope 12 includes a distal end portion 15 configured to emit and receive light transmitted therethrough from the light source 16 and to receive light reflected from the environment (e.g., tissue). The reflected light is transmitted through the endoscope and through a proximal end 17 that is coupled to the camera assembly 30.

The camera assembly 30 includes a housing 32 having a proximal end portion 32a and a distal end portion 32b with an opening 32c. The camera assembly 30 includes a connector 33 configured to releasably couple the endoscope 12 to the camera assembly 30, namely, the proximal end 17 to the housing 32. The connector 33 may be a spring-loaded locking connector that locks the endoscope 12 to the camera assembly 30 until released. The camera assembly 30 also includes a user interface 34 having one or more buttons for controlling the camera assembly 30, such as zoom, brightness, focus, etc.

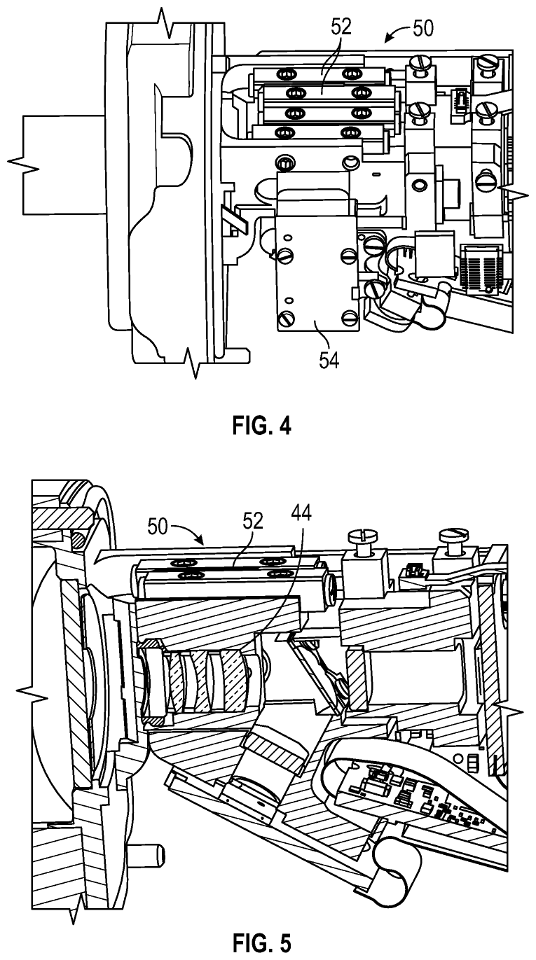

With reference to FIGS. 3-5, the camera assembly 30 includes a white (e.g., visible) light (VIS) sensor 36 and an IR sensor 38 and is configured to separate and transmit white light to the VIS sensor 36 and fluorescence IR light to the IR sensor 38. The VIS sensor 36 and the IR sensor 38 may be a complementary metal oxide semiconductor (CMOS) image sensors having any desired resolution, which in embodiments may be 4K, UHD, etc.

The camera assembly 30 further includes a cold mirror 46, which is a specialized dielectric mirror, which acts as a dichroic filter or beamsplitter, that reflects most or all the visible light along a visible light path while efficiently transmitting IR fluorescence light along IR light path. The cold mirror 46 may include a plurality of dielectric coatings disposed in a multi-layer configuration.

The light from the endoscope 12 is transmitted along a light path into the camera assembly 30. The camera assembly 30 also includes a focus group 44 having one or more lenses. The focus group 44 is configured to focus the light on the VIS sensor 36 and the IR sensor 38. This is accomplished by moving the focus group 44 longitudinally along the light path using any suitable drive mechanism (e.g., piezoelectric actuators).

The focus group 44 is supported on a sliding mechanism 50, which may include one or more crossed-roller bearings 52. The crossed-roller bearing 52 includes two sets of bearings and races disposed at right angles to each other. Bearings may be cylindrical bearings or rollers, which are mounted along the length of a rail in a carriage. The bearing may be held in place with a cage, preventing roller-to-roller contact, reducing friction and wear, and preventing jamming. The crossed-roller bearing 52 provides for more accurate movement and larger weight-bearing capacity for linear motion than other commonly used friction-reducing devices such as ball bearings and traditional linear rails. The cross-roller bearing 52 may also support moment loads, radial forces, or tilting loads. The crossed-roller bearing 52 also occupies less volume inside the housing 32 of the camera assembly 30 allowing for miniaturization of the camera assembly 30.

The focus group 44 is also coupled to a linear actuator 54, which may be a piezoelectric motor. In particular, the linear actuator 54 may have a piezoelectric element (e.g., rod) that converts electrical fields into mechanical strain thereby resulting in linear motion of the focus group 44. The piezoelectric motor is a direct drive actuator and as a result has zero backlash unlike conventional motorized focus mechanisms. Furthermore, the linear actuator 54 utilizes less components such as a drive screw, a nut, an anti-backlash mechanism, etc. and provides for maximum mechanical efficiency. As a result, the linear actuator 54 occupies less volume inside the housing 32 of the camera assembly 30. In addition, the piezoelectric element provides for precise movement in a stepwise manner, e.g., less than one micron.

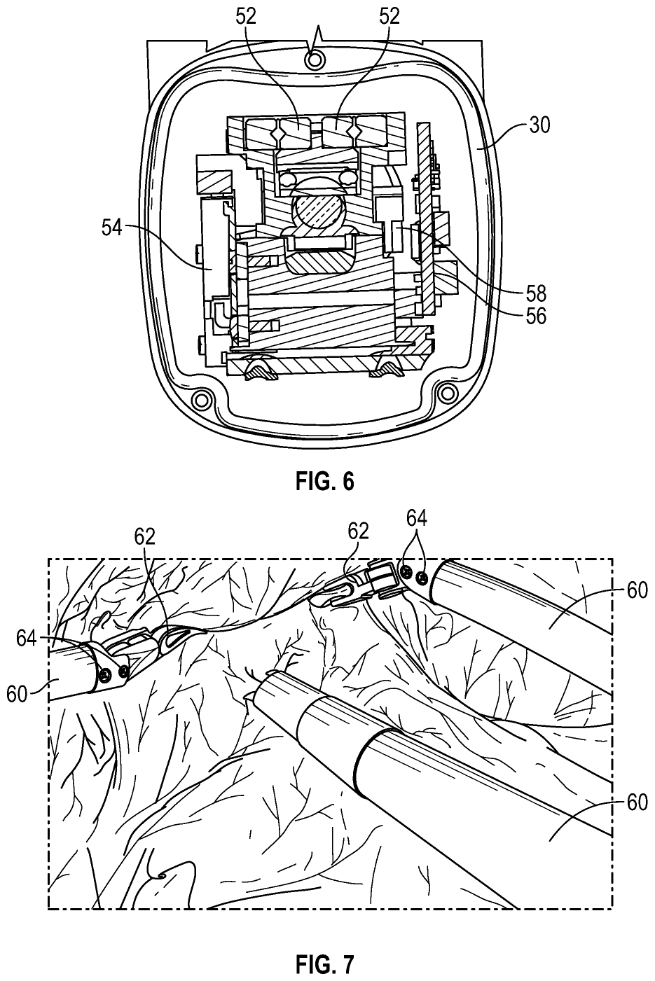

The focus group 44 may be coupled to the sliding mechanism 50 and to the linear actuator 54 using any suitable brackets, fasteners etc. and may be coupled in any suitable configuration, e.g., on opposing sides of the focus group 44 or such that the sliding mechanism 50 and the linear actuator 54 are adjacent to each other as shown in FIG. 6.

With continued reference to FIG. 6, position of the focus group 44 may be tracked using a variety of sensors, such as linear magnetic encoder 56 disposed on one side of the focus group 44. A magnet 58 is coupled to the focus group 44, such that as the focus group 44 is moved longitudinally by the linear actuator 54. As the magnet 58 is moved longitudinally along the linear magnetic encoder 56 positional information of the magnet 58 is detected by the magnetic encoder 56 based on the changes in the magnetic field and converts them into position signals.

The linear actuator 54 and magnetic encoder 56 are coupled to a controller 59 configured to control the linear actuator 54 in a closed loop manner based on sensor signals from the magnetic encoder 56. In embodiments, the controller 59 may be configured to control the linear actuator 54 using open loop control schemes as well.

The controller 59 may be any suitable processor operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be configured to perform operations, calculations, and/or set of instructions described in the disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be any logic processor (e.g., control circuit) adapted to execute algorithms, calculations, and/or set of instructions described herein.

The camera assembly 30 may be operated in a plurality of modes. The modes may be selected through the user interface 34 or may be executed by the controller 59 automatically depending on the operational state of the camera assembly 30. A homing mode is initiated at startup, during which the linear actuator 54 moves the focus group 44 between mechanical limits to define an index and calibrate the magnetic encoder 56 and the linear actuator 54. The focus range for the focus group 44 may be from about 1 mm to about 5 mm. After calibration, the linear actuator 54 may move the focus group 44 to a nominal position.

In one mode, the linear actuator 54 may be controlled using a velocity loop. The user may enter the velocity loop mode by pressing and holding and/or toggling a button on the user interface 34. During velocity loop mode, the linear actuator 54 is moved at a constant preset velocity. In a position loop mode, the linear actuator 54 is deactivated unless the magnetic encoder 56 senses movement of the camera assembly 30. In response to detecting movement, the linear actuator 54 is activated to compensate for the deviation of the focus group 44 due to the movement of the camera assembly 30.

In an auto-focus mode, the linear actuator 54 is moved at the fastest possible speed to move the focus group 44 to maintain focus of the image. In a tracking mode, the linear actuator 54 moves the focus group 44 to maintain focus on one or more surgical instruments 60 as shown in FIG. 7. In particular, the camera assembly 30 is configured to execute an autofocus tracking algorithm using machine learning or other image processing techniques to identify and track surgical instruments 60.

The surgical instruments 60 may be any suitable instrument, such as graspers, vessel sealers, directors, etc. In embodiments, the surgical instruments 60 may include fiducial markers 64, such as dots, patterns, etc. that are used to identify location of end effectors 62 of the surgical instruments 60. As the surgical instruments 60 are moved during surgery, the surgical instruments 60 and, in particular, the end effector 62 may go out of focus. As a result, the surgeon needs to stop the surgery to refocus and/or calibrate the camera assembly 30. The tracking algorithm keeps the end effectors 62 and/or portions of the surgical instruments 60 having the fiducial markers 64 in focus by using a closed loop adaptive position algorithm which adjusts the position of the focus group 44 via the linear actuator 54 which is controlled by the controller 59. The algorithm may be embodied as software instructions, executable by a controller 59.

In embodiments, the end effectors 62 and/or distal portions of the surgical instruments 60 may include a fluorescent material, which may be used to form one or more components of the end effector 62. The material may be a coating having a fluorescent dye, such as ICG. In further embodiments, the fiducial markers 64 may also include a fluorescent dye. Using a fluorescent dye makes the end effectors 62 easily detectable by the IR sensor 38, rather than using the VIS sensor 36 to identify and track the end effectors 62 and/or fiducial markers 64.

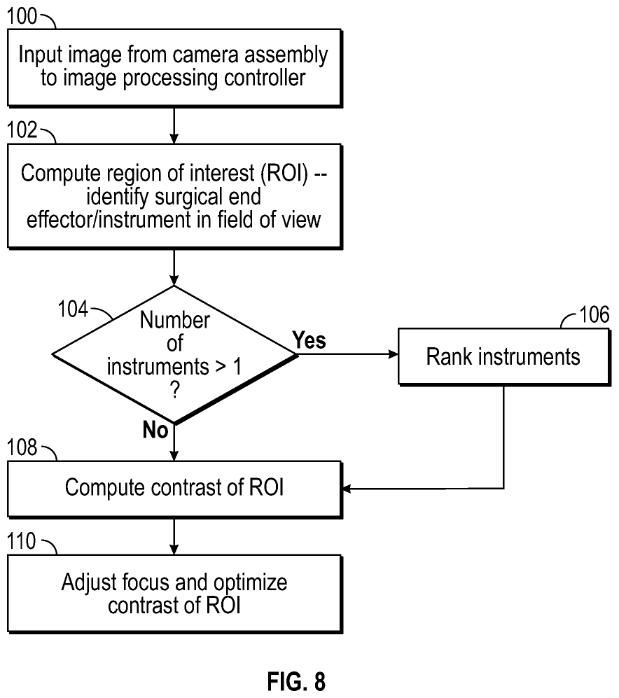

With reference to FIG. 8, a method for operating the control assembly 30 the tracking mode. At step 100, the camera assembly 30 captures an image. In embodiments, the camera assembly 30 may capture images at any suitable rate from about 30 Hz to about 144 Hz, and in further embodiments at about 60 Hz. The captured image or frame is provided to the controller 59, which at step 102, determines a region of interest 66 (FIG. 7). The ROI 66 may be determined using a computer vision algorithm derived from machine learning techniques, such as a deep neural network trained to recognize and identify type, position, orientation, operational state of the surgical instruments 60, the end effectors 62, and/or fiducial markers 64 in the field of view of the endoscope 12. The ROI 66 may also be any region where surgical activity is taking place, e.g., cutting, dissecting, energy application, grasping, stapling, etc. Thus, the ROI 66 is adaptive and may be located anywhere within the field of view of the endoscope 12 rather than one or more stationary positions, e.g., center.

Detecting the ROI 66 using machine learning may include direct activity localization. The computer vision algorithm may be provided labeling of active areas, such that the computer vision algorithm is trained to detect those events and their spatial location in the image. Detection of the surgical instruments 60 may be done by training the computer vision algorithm to detect the end effectors 62 and/or fiducial markers 64 in the image.

In embodiments, any other suitable autofocus techniques and systems may be used to detect the ROI 66, such as phase detection autofocus, which utilize a phase-detect sensor assembly to determine whether the image is in focus and automatically adjust the focus group 44.

In embodiments, where multiple surgical instruments 60 are detected at step 104, the computer vision algorithm at step 106 is configured to rank the surgical instrument 60 based on their relative importance during the procedure. The ranking order may be adjusted by the surgeon prior to or during the procedure. The ranking order may be automatically determined by the controller 59 based on a variety of factors (e.g., number of movements and activation of each of the surgical instruments 60). After identifying the most important surgical instrument 60, the ROI 66 is identified around that surgical instrument 60.

The computer vision algorithm may be a deep learning neural network for classifying images (i.e., extract contextual data) and may include a convolutional neural network (CNN) and/or a recurrent neural network. Generally, a deep learning neural network includes multiple hidden layers. The deep learning neural network may leverage one or more CNNs to classify one or more images, taken by the endoscope 12. In various methods, one or more CNNs may have a different amount of classification from each other. For example, a first CNN may have a five-class CNN, and a second CNN may have a six-class CNN. The deep learning neural network may be executed on any suitable controller.

Once the ROI 66 is determined, at step 108, the controller 59 computes the contrast for the ROI 66. Contrast may be computed by determining the difference between highest and lowest intensity values of the image and/or determining a histogram of the ROI 66. At step 110, the controller 59 signals the linear actuator 54 to adjust the focus group 44 to achieve a focus that optimizes the contrast of the ROI 66 while maintaining the ROI 66 in focus. Optimizing the contrast may include maximizing contrast.

While several embodiments of the disclosure have been shown in the drawings and/or described herein, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope of the claims appended hereto.

1. An endoscope camera assembly comprising:
a focus group including at least one lens, the focus group movable along a light path;
a sensor configured to receive light and to capture an image; and
a controller configured to:
identify a surgical instrument in the image;
determine a region of interest in the image based on a location of the surgical instrument in the image;
compute contrast of the region of interest; and
move the focus group to optimize contrast of the region of interest.

2. The endoscope camera assembly according to claim 1, wherein the controller is further configured to adjust focus of the region of interest.

3. The endoscope camera assembly according to claim 1, wherein the controller is further configured to determine the region of interest by identifying at least one of a surgical instrument, an end effector, or a fiducial marker.

4. The endoscope camera assembly according to claim 3, further comprising:
an infrared sensor configured to receive near infrared fluorescent light reflected from a fluorescent material of at least one of the surgical instrument, the end effector, or the fiducial marker, wherein the controller is further configured to determine the region of interest based on the near infrared fluorescent light.

5. The endoscope camera assembly according to claim 1, wherein the controller is further configured to identify a plurality of surgical instruments.

6. The endoscope camera assembly according to claim 5, wherein the controller is further configured to assign a rank to each surgical instrument of the plurality of surgical instruments.

7. The endoscope camera assembly according to claim 6, wherein the controller is further configured to determine the region of interest based on a surgical instrument of the plurality of surgical instruments which has a highest rank.

8. The endoscope camera assembly according to claim 1, further comprising:
a sliding mechanism coupled to the focus group; and
a linear actuator coupled to the focus group and configured to move the focus group along the sliding mechanism.

9. The endoscope camera assembly according to claim 8, wherein the sliding mechanism includes at least one crossed-roller bearing.

10. The endoscope camera assembly according to claim 9, wherein the linear actuator is a piezoelectric motor.

11. The endoscope camera assembly according to claim 10, further comprising:
a magnet coupled to the focus group; and
a linear magnetic encoder disposed adjacent to the focus group and the magnet, the linear magnetic encoder configured to measure a position of the focus group.

12. The endoscope camera assembly according to claim 11, wherein the controller is coupled to the linear actuator and the linear magnetic encoder, the controller is further configured to control the linear actuator based on the measured position of the focus group.

13. A method for controlling an endoscope camera assembly, the method comprising:
receiving light through a focus group including at least one lens, the focus group movable along a light path;
capturing an image at a sensor configured to receive light;
identifying a surgical instrument in the image;
determining a region of interest in the image based on a location of the surgical instrument in the image;
computing contrast of the region of interest; and
moving the focus group to optimize contrast of the region of interest.

14. The method according to claim 13, further comprising:
moving the focus group to adjust focus of the region of interest.

15. The method according to claim 13, wherein determining the region of interest further includes identifying at least one of a surgical instrument, an end effector, or a fiducial marker.

16. The method according to claim 13, further comprising:
identifying a plurality of surgical instruments.

17. The method according to claim 16, further comprising:
assigning a rank to each surgical instrument of the plurality of surgical instruments.

18. The method according to claim 17, wherein determining the region of interest is based on a surgical instrument of the plurality of surgical instruments which has a highest rank.

19. An endoscope camera assembly comprising:
a focus group including at least one lens;
a sliding mechanism coupled to the focus group configured to move the focus group along a light path;
a linear actuator coupled to the focus group configured to move the focus group along the sliding mechanism;
a magnet coupled to the focus group;
a linear magnetic encoder disposed adjacent the focus group and the magnet, the linear magnetic encoder configured to measure a position of the focus group;
a sensor configured to receive light and to capture an image; and
a controller configured to:
identify a surgical instrument in the image;
determine a region of interest in the image based on a location of the surgical instrument in the image;
compute contrast of the region of interest; and
move the focus group to optimize contrast of the region of interest.

20. The endoscope camera assembly according to claim 19, wherein the controller is coupled to the linear actuator and the linear magnetic encoder, the controller is further configured to control the linear actuator based on the measured position of the focus group.
